(19) United States
(12) Patent Application Publication
(10) Pub. No.: US 2007/0243637 A1
(43) Pub. Date: Oct. 18, 2007

(54) Process and device for determining the concentrations of ligands

(75) Inventors: Sonja Mohry, Freiburg (DE); Holger Klapproth, Freiburg (DE); Mirko Lehmann, Freiburg (DE); Ingo Freund, Freiburg (DE)

Correspondence Address: THE WEBB LAW FIRM, P.C., 700 KOPPERS BUILDING, 436 SEVENTH AVENUE, PITTSBURGH, PA, 15219, US

(73) Assignee: Micronas GmbH, Freiburg i.Br. (DE)

(21) Appl. No.: 11/784,768

(22) Filed: Apr. 9, 2007

(30) Foreign Application Priority Data
Apr. 10, 2006 (EP) 06 007 478.8

(51) Int. Cl. G01N 33/543; C12M 3/00

(52) U.S. Cl. 436/518; 435/287.2

(57) ABSTRACT

In a process for determining the concentrations of ligands (9, 10) in a sample to be analyzed, ligands (9) of a first ligand type are bond-specific for receptors (6) of a first receptor type, and ligands (10) of a second ligand type are bond-specific for receptors (8) of a second receptor type. A first receptor (6) of the first receptor type is immobilized at least one first test site (5), and a second receptor (8) of the second receptor type is immobilized at a second test site (7) on a substrate (23). A third receptor (11) of the second receptor type is mixed with the sample in such a way that it is present in a specified concentration in the mixture that is obtained in this manner. The mixture is caused to contact the substrate in such a way that at least one ligand (9) of the first ligand type can bind to the first receptor (6) and ligands (10) of the second ligand type can bind to the second receptor (8) and to the third receptor (11). Thereupon components of the mixture that are not bound to an immobilized receptor (6, 8) are removed from the substrate. Thereupon a first measurement signal that is dependent on the concentration of the ligand (9) bound to the first receptor (6) and a second measurement signal that is dependent on the concentration of the ligand (10) bound to the second receptor (8) are generated. With the aid of the first measurement signal the concentration of the ligand (9) of the first ligand type in the sample is determined and, with the aid of the second measurement signal and of the concentration of the third receptor (11) in the mixture, the concentration of the ligands (10) of the second ligand type in the sample is determined.

The invention relates to a process for determining the concentrations of ligands in a sample to be analyzed, in which ligands of a first ligand type are bond-specific for receptors of a first receptor type, and ligands of a second ligand type are bond-specific for receptors of a second receptor type, in which at least one first receptor of the first receptor type is immobilized at least one first test site, and at least one second receptor of the second receptor type is immobilized at least one second test site on a substrate. The invention further relates to a device for determining the concentrations of ligands of various ligand types in a sample to be analyzed, said device having a measurement chamber that has at least one inlet opening and one outlet opening and that has a substrate on which a first receptor of a first receptor type is immobilized at a first test site, and a second receptor of a second receptor type is immobilized at a second test site, in which the first receptor type is bond-specific for the first ligand type and the second receptor type is bond-specific for the second ligand type, in which a first sensor for acquiring a first measurement signal that is dependent on the concentration of the ligand bound to the first receptor is assigned to the first test site, and a second sensor for acquiring a second measurement signal that is dependent on the concentration of the ligand bound to the second receptor is assigned to the second test site.

A device of this type is disclosed in U.S. Pat. No. 6,197,503 B1. It has a flow-through measurement chamber that is defined on its lower side by a generally plate-shaped substrate upon which there are located a plurality of test sites at which receptors are immobilized. The receptors are each bond-specific for a particular ligand contained in a sample that is to be analyzed. A semiconductor chip that has one optical sensor at each of the individual test sites is provided on the back side of the substrate. In order to stimulate the emission of luminescence radiation as a function of the binding of the ligands at the receptors, the device has an optical radiation source in whose irradiation zone the receptors are located. In order to measure the concentration of the ligands, the sample is supplied to the measurement chamber through an inlet opening in such a way that the ligands contained in the sample can bind to the receptors. After a predetermined exposure time has passed, components of the sample that have not bound to a receptor are removed from the measurement chamber. The exposure time is selected in such a way that, after the unbound components are removed, only some of the binding sites of a receptor are bound to the ligands that are bond-specific for the given receptor. Thus, the ratio of bound binding sites to free binding sites is dependent on the concentration of the respective ligands in the sample.

In a further process step, a solution that contains detection antibodies that are marked with a marker is fed into the measurement chamber. The detection antibodies are bond-specific for the ligands, and they bind to these ligands. The solution is then removed from the measurement chamber in order to irradiate the test sites with the excitation radiation with the aid of the radiation source. The detection antibodies that are indirectly bound to the receptors by means of the ligands are excited by the excitation radiation, causing them to emit a luminescence radiation whose wavelength differs from the wavelength of the excitation radiation. With the aid of optical sensors that are sensitive to the luminescence radiation and that are insensitive to the excitation radiation, a measurement signal that is dependent on the luminescence radiation and thus on the concentration of the ligands in the sample is generated for each test site.

The device has the disadvantage that it only permits the simultaneous determination of the individual concentration values of the various ligands contained in the sample in a limited concentration range. However, in certain samples, for example blood samples, the ligands contained in the sample may have concentration differences of up to six decimal places. At least two tests are performed so that with such a sample a concentration value can be determined both for the ligands having the lowest concentration as well as for the ligands having the highest concentration without all of the receptor binding sites that are present binding to one ligand at one of the test sites, thereby limiting the corresponding test value. In a first test to determine the concentration of the ligands that have the highest concentration value, a low exposure time is selected, and in a second test to determine the concentration of the ligand having the lowest concentration value, a substantially larger exposure time is selected than in the first test. The disadvantage of this is that a new semi conductor chip is required for each test, which is why the measurement remains relatively expensive and labor-intensive. Another disadvantage is that a correspondingly large sample size is required. When blood samples that are taken from a patient's finger using a puncturing device are tested, this can mean that blood must be taken from the finger at least two locations, which is particularly problematical with children.

The object of the invention is therefore to create a device and a process of the type stated above that permit the simple, fast, and economical measurement of the concentration of a plurality of ligands that are suspected to be present in a sample that is to be tested.

This object is accomplished with respect to the process in such a way that at least one third receptor of the second receptor type is mixed with the sample in such a way that it is present in a specified concentration in the mixture that is obtained in this manner, that the mixture is caused to contact the substrate in such a way that at least one ligand of the first ligand type can bind to the first receptor and ligands of the second ligand type can bind to the second receptor and to the third receptor, that thereafter components of the mixture that are not bound to an immobilized receptor are removed from the substrate, that then a first measurement signal that is dependent on the concentration of the ligand bound to the first receptor and a second measurement signal that is dependent on the concentration of the ligand bound to the second receptor are generated, and that with the aid of the first measurement signal the concentration of the ligand of the first ligand type in the sample is determined and with the aid of the second measurement signal and of the concentration of the third receptor in the mixture, the concentration of the ligands of the second ligand type in the sample is determined.

In this process, the third receptor binds to ligand in such a way that it can no longer bind to the second receptor. This is advantageous in that it makes it possible to determine in a single test the concentrations of the individual ligands in samples that contain a plurality of ligands whose concentrations differ greatly from each other. The concentration of the third receptor in the mixture comprising the sample and the third receptor and the exposure time during which the mixture is in contact with the receptors are thereby matched to the concentration ranges to be tested for the individual ligands in such a way that, after the components of the mixture that are not bound to a receptor are removed, at all of the test sites only some of the binding sites bind receptors to a ligand, provided that this ligand is contained in the sample in the concentration range to be tested. The concentration of the second ligand can be calculated from the second measurement signal and the known concentration of the third receptor in the mixture using the law of mass action. The concentration of the third receptor in the mixture is preferably selected in such a way that it corresponds to a limit value of the concentration of the second ligand, from which a determination such as “good/bad” or “ill/healthy” can be derived. Using the process of the invention, ligand concentrations in a range from g/L to μg/L can be detected concurrently in a flow cell or similar measurement chamber.

In a preferred embodiment of the invention, a detection antibody that is bond-specific for the first ligand and that is marked with a first marker can be brought into contact with the first test site, then markers that are not bound to an immobilized receptor are removed from the substrate, and then the first measurement signal is generated as a function of the concentration of the first marker. The concentration of the first ligand can thus be measured with the aid of a sandwich ELISA.

In a preferred embodiment of the invention, a detection antibody that is bond-specific for the ligands of the second ligand type and that is marked with a second marker is brought into contact with the second test site, then markers that are not bound to an immobilized receptor are removed from the substrate, and then the second measurement signal is generated as a function of the concentration of the second marker and the concentration of the third receptor in the mixture. The concentration of the first ligand can thus also be measured with the aid of a sandwich ELISA.

It is advantageous if the first marker and/or second marker is/are an enzyme, if the enzyme is brought into contact during the acquisition of the measurement signals with the least two chemicals between which a chemical redox reaction occurs when the enzyme is present, and if the first measurement signal and/or the second measurement signal is/are generated by measuring a redox potential. The redox potential may be measured in this process with the aid of an ISFET.

In a preferred embodiment, of the invention the first marker and/or second marker is/or irradiated during the acquisition of the measurement signals with an excitation radiation that excites the marker(s) to emit a luminescence radiation, and the first measurement signal and/or the second measurement signal is/are generated by measuring the luminescence radiation of the respective marker. In this process, the excitation radiation may be measured with the aid of a light source, for example a light-emitting diode and/or a xenon lamp. The same excitation radiation is preferably used for the first and second markers. However, it is also conceivable that the first and second markers are different dyes, such as Cy3 and Cy5, and that these dyes are excited at different wavelengths.

It is advantageous if the first test site and/or the second test site is/are brought into contact during the acquisition of the measurement signals with a chemiluminescence substrate in which a luminescence radiation is stimulated as a function of the binding of the ligands of the first ligand type to the first receptor and/or as a function of the binding of the ligands of the second receptor type to the second receptor, and if the first measurement signal and/or the second measurement signal is/are generated by measuring the luminescence radiation at the respective test site. In this way, the luminescence radiation is generated by chemical means without an excitation radiation.

With respect to the device, the object stated above is achieved when at least one non-immobilized third receptor of the second receptor type is disposed in the measurement chamber in such a way that upon contact with the sample to be analyzed this receptor and the sample form a mixture that contains the third receptor in the specified concentration, and when the second sensor is connected to an evaluation device that is designed to determine the concentration of the second ligand in the sample from the second measurement signal and the specified concentration of the third receptor in the mixture.

Thus, during and/or after its entry into the measurement chamber the sample is mixed with a receptor of the second receptor type that is bond-specific for the second ligand, so that the second ligand is captured by the third receptor, and the available concentration of the this [sic] ligand is thereby reduced. As already explained with the process, for a sample that contains a plurality of ligands whose concentrations differ greatly, the concentrations of the individual ligands can be determined with just a single test.

It is advantageous if the third receptor is stabilized in the test chamber in a gel-like, paste-like, or solid form, preferably in such a way that it adheres to one of the walls that define the measurement chamber. This makes the device especially easy to handle. The system for stabilizing the competitor preferably comprises at least one non-reducing disaccharide and at least one protein or polypeptide of the LEA class. The non-reducing disaccharide may be selected from the group comprising trehalose (D-glucopyranosyl-D-glucopyranose), sucrose (β-D-fructofuranosyl-α-D-glucopyranoside) and derivatives thereof. A stabilization system of this type is described in WO 2004/004455 A2. The competitor that is present in stabilized form can be stored for a relatively long time.

The above object is also achieved with respect to the device when the device has a mixing device that is used to mix the sample with at least one third receptor of the second receptor type and that is connected to a feed opening for the sample and to a receiving space containing the third receptor, when the mixing device has a discharge opening that is used for the mixture formed from the sample and the third receptor and that is connected to the inlet opening of the measurement chamber, when the mixing device is designed in such a way that the third receptor is present in a specified concentration in the mixture, and when the second sensor is connected to an evaluation device that is designed to determine the concentration of the second ligand in the sample from the second measurement signal and the concentration of the third receptor in the mixture.

In this solution, therefore, the sample is already mixed with the aid of the mixing device with a receptor of the second receptor type that is bond-specific for the second ligand before the sample enters the measurement chamber. This permits a particularly homogeneous distribution of the receptor of the second receptor type in the sample. In this way, the concentration of the second ligand can be measured with high precision.

It is advantageous if the third receptor is stabilized in the receiving space in a gel-like, paste-like, or solid form, preferably in such a way that it adheres to one of the walls that define the receiving space, and that the receiving space is embodied as a flow-through mixing chamber by which means the feed opening for the sample is connected to the inlet opening in the measurement chamber in order to dissolve the third receptor in the sample. This makes the device easy to handle.

In a preferred embodiment of the invention, the flow-through measurement chamber has a mixer structure that is designed in such a way that the mixture is preferably diverted into directions that alternately oppose each other when it flows through the flow-through mixing chamber, and the mixer structure is located between the third receptor, which is present in a gel-like, paste-like, or solid form, and the inlet opening of the measurement chamber. This mixer may be a so-called Möbius mixer that can be produced with extremely compact dimensions using the methods employed in microsystem technology.

In a preferred embodiment of the invention, the sensors are optical sensors, and the first receptor for detecting the first luminescence radiation as a function of the binding of the ligands of the first ligand type to the first receptor is preferably located directly on the first sensor, and/or the second receptor for detecting the second luminescence radiation that is generated as a function of the binding of the ligands of the second ligand type to the second receptor is preferably located directly on the second sensor. The luminescence radiation that is generated at the receptors may then pass directly into the sensor(s) without taking an indirect route through a collection lens.

The device may be a component of a kit of one of Claims 13 to 18, which in addition to the device may have:

a detection antibody that is bond-specific for the first and/or second ligand and that is marked with an enzyme or similar marker,

at least two chemicals between which a chemical redox reaction occurs upon contact with the enzyme located on the detection antibody, and/or

a chemiluminescence substrate in which a chemical reaction that releases luminescence radiation is triggered upon contact with the enzyme located on the detection antibody, and/or

a radiation source that is located on the first test site and/or second in order to emit the luminescence radiation.

The marker, the chemiluminescence substrate, and/or the chemicals may be transported to the measurement chamber by means of a suitable feed device, for example a micropump or a pipette.

A typical embodiment of the invention is explained in greater detail below on the basis of the drawing. The drawing shows:

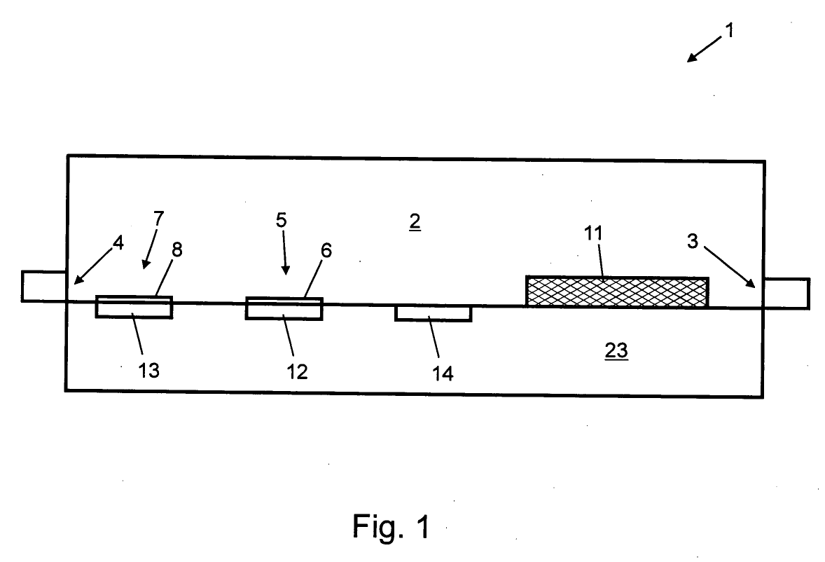

FIG. 1 a longitudinal cross-section through a first typical embodiment of a device for determining the concentrations of ligands contained in a sample to be analyzed,

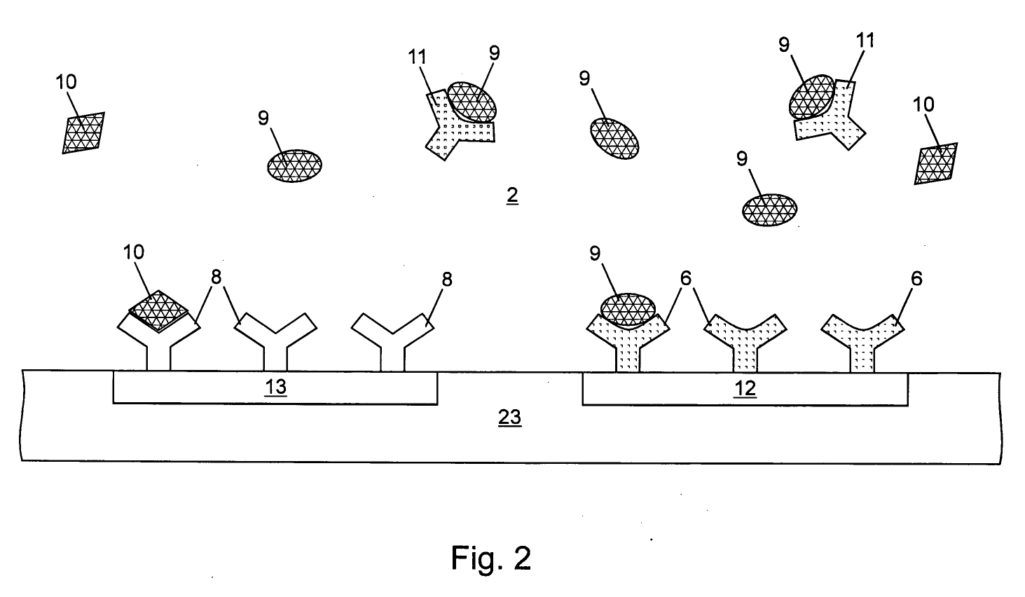

FIG. 2 a partial view of a measurement chamber device that is filled with a sample to be analyzed. The immobilized receptors and ligands contained in the sample are depicted schematically on one of the walls of the measurement chamber,

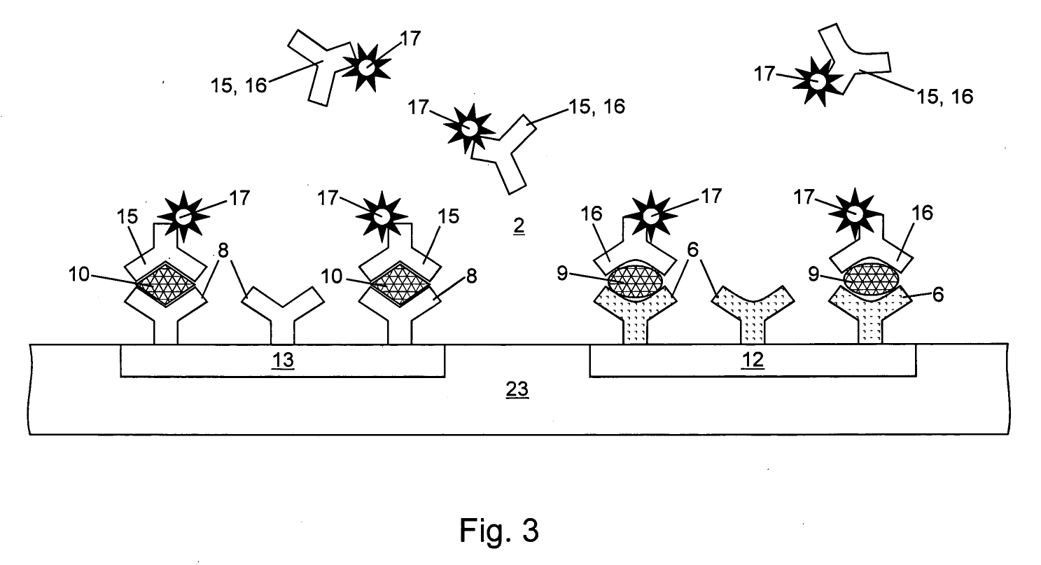

FIG. 3 A partial view of the measurement chamber following the marking of receptor-ligand complexes,

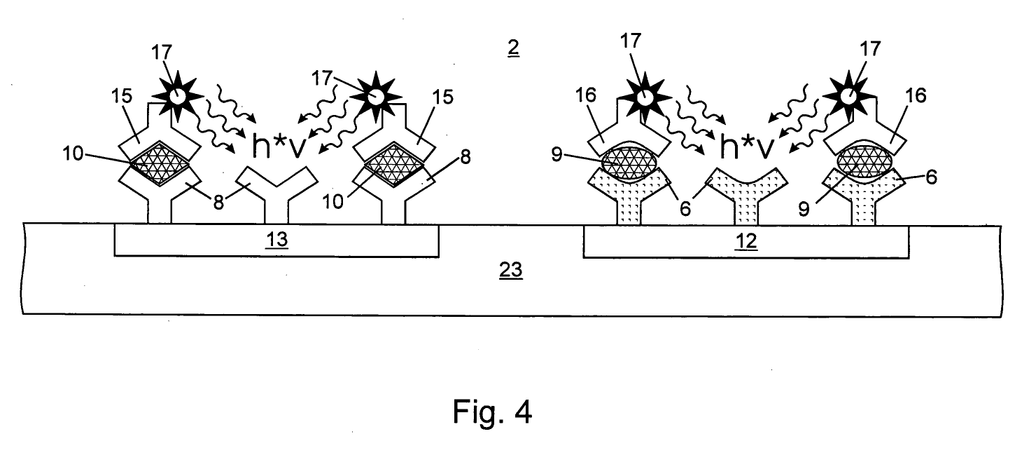

FIG. 4 a partial view of the measurement chamber after a chemiluminescence substrate has been fed into the measurement chamber, and

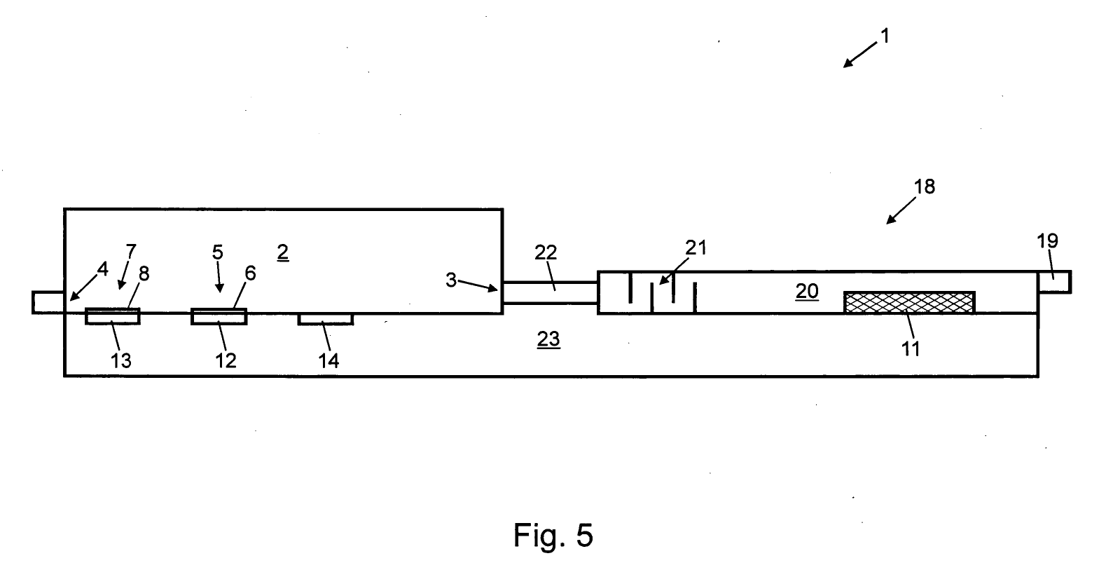

and FIG. 5 a longitudinal cross-section through a second typical embodiment of a device for determining the concentrations of ligands contained in a sample to be analyzed.

A device that is shown in complete form in FIG. 1 for determining the concentrations of at least two ligands 9, 10 in a sample that is to be analyzed has a measurement chamber 2 that is embodied as a flow cell and that has an inlet opening 3 and an outlet opening 4. One wall of the measurement chamber 2 is formed by a semiconductor substrate 23 on which at a first test site 5 at least one first receptor 6 of the first receptor type and, at a second test site 7 that is present at a lateral distance therefrom, at least one second receptor 8 of a second receptor type are immobilized. The first receptor 6 is bond-specific for a first ligand 9 contained in the sample to be analyzed; and the second receptor 8 is bond-specific for a second ligand 10 contained in the sample. The second ligand 10 has a greater concentration in the sample than the first ligand 9.

In addition, at least one third receptor 11 of the second receptor type, which is not immobilized and is present in lyophilized form, is located in the measurement chamber 2. The third receptor 11 adheres to one of the walls that define the measurement chamber 2. Upon contact with the aqueous sample that is to be analyzed, the third receptor 11 dissolves in the sample.

At the first test site 5 a first optical sensor 12 located directly below the first receptor 6, and at the second test site 7 a second optical sensor 13 located directly below the second receptor 8 are integrated into the semiconductor substrate 23. In addition, a third optical sensor 14 that is not covered by a receptor and that serves as a reference value sensor is located in the wall of the measurement chamber. Sensors 12, 13, and 14 may, for example, be photodiodes.

In order to determine the concentrations of ligands 9 and 10, the sample is first filled into the measurement chamber 2 through the Inlet opening 3 so that the entire volume of the measurement chamber 2 is filled with the sample. As soon as the third receptor 11 comes into contact with the sample, it dissolves in the sample. The dissolving operation may be accelerated by applying mechanical energy to the measurement chamber 2. The amount of the third receptor 11 is matched to the volume of the measurement chamber 2 in such a way that the mixture that is formed from the sample and the third receptor 11 contains the third receptor 11 in a specified concentration corresponding to a limit value that is to be measured if this receptor is completely and homogeneously dissolved in the sample.

After the measurement chamber 2 is filled with the sample, the process is held for a specified first period of time during which the third receptor 11 can dissolve in the sample. As can be seen in FIG. 2, the first period of time is selected so that ligands 9 and 10 contained in the sample are able to bind to receptors 6, 8, and 10 that are bond-specific for them. It can be clearly seen that only some of the available free binding sites of receptors 6 and 8 that are present at the individual test sites 5 and 7 bind to a ligand 9, 10.

After the specified first period of time is completed, a rinsing liquid that removes the components of the resulting mixture that are not bound to a receptor 6, 8 from the measurement chamber 2 is passed through the measurement chamber 2 via the feed opening 14, the inlet opening 3, and the outlet opening 4.

Then a solution that contains the detection antibodies 15, 16 is fed into the test chamber 2. A first detection antibody 15 is bond-specific for ligands 9 of the first binding type, and a second detection antibody 15 is bond-specific for ligands 10 of the second of ligand type. The detection antibodies 15, 16 are each marked with an enzyme 17, such as HRP. After the detection antibody 15, 16 has been fed into the measurement chamber 2, the process is held for a specified second period of time, which is selected such that nearly all of the first ligands 9 that are bound to the first receptor 6 bind to a first detection antibody 15 and nearly all of the second ligands 10 that are bound to the second receptor 8 bind to a second detection antibody 15 and are thereby indirectly marked with the enzyme 17 (FIG. 3).

After the specified period of time has been completed, rinsing liquid is once again passed through the measurement chamber 2 in order to remove detection antibodies 15, 16 that are not bound to a ligand 9, 10 from the measurement chamber 2.

Then a chemiluminescence substrate that contains hydrogen peroxide and a chemiluminescent substance, such as luminol, is fed into the measurement chamber 2 through the inlet opening 3. If the enzyme 17 comes into contact with the hydrogen peroxide, free oxygen radicals are cleaved off of the hydrogen peroxide, whereby the chemiluminescent substance decomposes chemically accompanied by the emission of luminescence radiation. The luminescence radiation is thus generated in the measurement chamber 2 as a function of the binding of the first ligand 9 to the first receptor 6 and as a function of the binding of the second ligand 10 to the second receptor 8.

The optical sensors 12, 13 are sensitive to the luminescence radiation and are disposed relative to the receptors 6, 8 in such a way that each of them only detects the luminescence radiation that is generated at the test site 5, 7 that is assigned to them, but not the luminescence radiation that is generated at the other test site 7, 5, respectively.

An evaluation device that is not shown in greater detail in the drawing is connected to the sensors 12, 13. It determines the concentrations of the first ligand 6 in the sample as a function of the measurement signal of the first sensor 12, and it determines the concentration of the second ligand 10 in the sample as a function of the measurement signal of the second sensor 13 and the known concentration of the competitor 15 in the mixture with the aid of the law of mass action. The evaluation device may be integrated as an electrical circuit into the substrate 23 or the wall of the measurement chamber 2.

In the embodiment shown in FIG. 5, the device 1 has a mixing chamber 2 that is located upstream from the measuring device 18 and that connects the inlet opening 3 to a feed opening 19. A receiving space 20 that contains the third receptor 11 in a stabilized form is located between the feed opening 19 and the inlet opening 3. The mixing device 13 is used to mix the sample homogeneously with the third receptor 11. To accomplish this, the sample is transported, with the aid of a pipette or a pump, for example, through the feed opening 19 into the receiving space 20. In the receiving space 20 the third receptor 11 is disposed relative to the feed opening 19 in such a way that the sample comes in contact with the third receptor 11 during and/or after it is introduced into the receiving space 20.

The mixing device 18 is designed in such a way that the third receptor 11 in the mixing chamber 2 is present in the mixture in a specified concentration that corresponds to the limit value to be measured. This is achieved when the measurement chamber 2 and the mixing device 18 have a predetermined volume, and when amount of the third receptor 11 that is stabilized in the receiving space 20 is selected such that the desired concentration is established when the third receptor 11 is mixed with the sample to form a mixture that has the specified volume.

Seen in the direction of flow downstream from the receiving space 20, the mixing device 18 has a mixer structure 21 that is configured as a Möbius mixer and that is connected at one of its ends to the receiving space 20, and at its other end, which has a discharge opening for the mixture formed from the sample and the third receptor 11, said mixing device is connected by means of a channel 22 to the inlet opening 3 of the mixing chamber 2.

The construction of the mixing chamber 2 essentially corresponds to that shown in FIG. 1, the difference being that a third receptor 11 is not disposed in the mixing chamber. Otherwise, the reader is directed to the description provided there.

1. A process for determining the concentrations of ligands in a sample to be analyzed, in which ligands of a first ligand type are bond-specific for receptors of a first receptor type, and ligands of a second ligand type are bond-specific for receptors of a second receptor type, in which at least one first receptor of the first receptor type is immobilized at least one first test site, and at least one second receptor of the second receptor type is immobilized at least one second test site on a substrate, in which at least one third receptor of the second receptor type is mixed with the sample in such a way that it is present in a specified concentration in the mixture that is obtained in this manner, in which the mixture is caused to contact the substrate in such a way that at least one ligand of the first ligand type can bond to the first receptor and ligands of the second ligand type can bind to the second receptor and to the third receptor, in which thereupon components of the mixture that are not bound to an immobilized receptor are removed from the substrate, in which thereupon a first measurement signal that is dependent on the concentration of the ligand bound to the first receptor and a second measurement signal that is dependent on the concentration of the ligand bound to the second receptor are generated, and in which with the aid of the first measurement signal the concentration of the ligand of the first ligand type in the sample is determined and with the aid of the second measurement signal and of the concentration of the third receptor in the mixture, the concentration of the ligands of the second ligand type in the sample is determined.

2. The process of claim 1, wherein a detection antibody that is bond-specific for the ligands of the first ligand type and that is marked with a first marker is brought into contact with the first test site and thereafter markers that are not bound to an immobilized receptor are removed from the substrate, and the first measurement signal is then generated as a function of the concentration of the first marker.

3. The process of claim 1, wherein a detection antibody that is bond-specific for the ligands of the second ligand type and that is marked with a second marker is brought into contact with the second test site and thereafter markers that are not bound to an immobilized receptor are removed from the substrate, and the second measurement signal is then generated as a function of the concentration of the second marker and the concentration of the third receptor in the mixture.

4. The process of claim 1, wherein the first marker and/or second marker is/are an enzyme, the enzyme is brought into contact during the acquisition of the measurement signals with the least two chemicals between which a chemical redox reaction occurs when the enzyme is present, and the first measurement signal and/or the second measurement signal is/are generated by measuring a redox potential.

5. The process of claim 1, wherein the first marker and/or second marker is/are irradiated during the acquisition of the measurement signals with an excitation radiation that stimulates the marker(s) to emit a luminescence radiation, and the first measurement signal and/or the second measurement signal is/are generated by measuring the luminescence radiation of the respective marker.

6. The process of claim 1, wherein the first test site and/or second test site is/are brought into contact during the acquisition of the measurement signals with a chemiluminescence substrate in which a luminescence radiation is stimulated as a function of the binding of the ligands of the first ligand type to the first receptor and/or as a function of the binding of the ligands of the second receptor type to the second receptor, and the first measurement signal and/or the second measurement signal is/are generated by measuring the luminescence radiation at the respective test site.

7. A device for determining the concentrations of ligands of various ligand types in a sample to be analyzed, said device having a measurement chamber that has at least one inlet opening and one outlet opening and that has a substrate on which a first receptor of a first receptor type is immobilized at a first test site, and a second receptor of a second receptor type is immobilized at a second test site, in which the first receptor type is bond-specific for a first ligand type, and the second receptor type is bond-specific for a second ligand type, in which a first sensor for acquiring a first measurement signal that is dependent on the concentration of the ligand bound to the first receptor is assigned to the first test site, and a second sensor for acquiring a second measurement signal that is dependent on the concentration of the ligand bound to the second receptor is assigned to the second test site, wherein at least one non-immobilized third receptor of the second receptor type is disposed in the measurement chamber in such a way that when this receptor contacts the sample to be analyzed, a mixture that contains the third receptor in the specified concentration is formed, and the second sensor is connected to an evaluation device that is designed to determine the concentration of the second ligand in the sample from the second measurement signal and the specified concentration of the third receptor in the mixture.

8. The device of claim 7, wherein the third receptor is stabilized in the measurement chamber in a gel-like, paste-like, or solid form preferably in such a way that it adheres to one of the walls defining the measurement chamber.

9. A device for determining the concentrations of ligands of various ligand types in a sample to be analyzed, said device having a measurement chamber that has at least one inlet opening and one outlet opening and that has a substrate on which a first receptor of a first receptor type is immobilized at a first test site and a second receptor of a second receptor type is immobilized at a second test site in which the first receptor type is bond-specific for a first ligand type and the second receptor type is bond-specific for a second ligand type in which a first sensor for acquiring a first measurement signal that is dependent on the concentration of the ligand bound to the first receptor is assigned to the first test site and a second sensor for acquiring a second measurement signal that is dependent on the concentration of the ligand bound to the second receptor is assigned to the second test site, wherein, the device has a mixing device that is used to mix the sample with at least one third receptor of the second receptor type and that is connected to a feed opening for the sample and to a receiving space containing the third receptor, the mixing device has a discharge opening that is used for the mixture formed from the sample and the third receptor and that is connected to the inlet opening of the measurement chamber, the mixing device is designed in such a way that the third receptor is present in a specified concentration in the mixture, and the second sensor is connected to an evaluation device that is designed to determine the concentration of the second ligand in the sample from the second measurement signal and the concentration of the third receptor in the mixture.

10. The device of claim 9, wherein the third receptor is stabilized in the receiving space in a gel-like, paste-like, or solid form, preferably in such a way that it adheres to one of the walls that define the receiving space, and the receiving space is embodied as a flow-through mixing chamber by which means the feed opening for the sample is connected to the inlet opening in the measurement chamber in order to dissolve the third receptor in the sample.

11. The device of claim 9, wherein the flow-through measurement chamber has a mixer structure that is designed in such a way that the mixture is preferably diverted into directions that alternately oppose each other when it flows through the flow-through mixing chamber, and the mixer structure is located between the third receptor, which is present in a gel-like, paste-like, or solid form, and the inlet opening of the measurement chamber.

12. The device of claim 7, wherein the sensors are optical sensors, and the first receptor for detecting the first luminescence radiation as a function of the binding of the ligands of the first ligand type to the first receptor is preferably located directly on the first sensor, and/or the second receptor for detecting the second luminescence radiation that is generated as a function of the binding of the ligands of the second ligand type to the second receptor is preferably located directly on the second sensor.

13. A kit for determining the concentrations of ligands, comprising:
a device according to claim 7, in which the first sensor is embodied as a sensor for measuring a redox potential,
a detection antibody that is bond-specific for ligands of the first ligand type and that is marked with an enzyme, and
at least two chemicals between which a chemical redox reaction occurs upon contact with the enzyme.

14. A kit for determining the concentrations of ligands, comprising:
a device according to claim 7, in which the second sensor is embodied as a sensor for measuring a redox potential,
a detection antibody that is bond-specific for ligands of the second ligand type and that is marked with an enzyme, and
at least two chemicals between which a chemical redox reaction occurs upon contact with the enzyme.

15. A kit for determining the concentrations of ligands, comprising:
a device according to claim 7,
a detection antibody that is bond-specific for ligands of the first ligand type and that is marked with an enzyme, and
a chemiluminescence substrate in which a chemical reaction that releases luminescence radiation for which the first sensor is sensitive is triggered upon contact with the enzyme.

16. A kit for determining the concentrations of ligands, comprising:
a device according to claim 7,
a detection antibody that is bond-specific for ligands of the second ligand type and that is marked with an enzyme, and
a chemiluminescence substrate in which a chemical reaction that releases luminescence radiation for which the second sensor is sensitive is triggered upon contact with the enzyme.

17. A kit for determining the concentrations of ligands, comprising:
a detection antibody that is bond-specific for ligands of the first ligand type and that is marked with a marker that can be excited by irradiation with an excitation radiation to emit a luminescence radiation,
a device according to claim 7, in which the first sensor is sensitive for the luminescence radiation and is insensitive for the excitation radiation, and
a radiation source that is located on the first test site in order to emit the luminescence radiation.

18. A kit for determining the concentrations of ligands, comprising:
a detection antibody that is bond-specific for ligands of the first ligand type and that is marked with a market that can be exited by irradiation with an excitation radiation to emit a luminescence radiation,
a device according to claim 7, in which the second sensor is sensitive for the luminescence radiation and is insensitive for the excitation radiation, and
a radiation source that is located on the second test site in order to emit the luminescence radiation.
